(19) United States
(12) Patent Application Publication
(10) Pub. No.: US 2025/0180421 A1
(43) Pub. Date: Jun. 5, 2025

(54) TEXTILE-BASED LARGE-AREA PRESSURE SENSING ARRAYS

(71) Applicants: ISTANBUL TEKNIK UNIVERSITESI, Istanbul (TR); MARMARA UNIVERSITESI, Istanbul (TR)

(72) Inventors: Cagatay GUMUS, Istanbul (TR); Kadir OZLEM, Bursa (TR); Fidan KHALILBAYLI, Istanbul (TR); Omur Fatmanur ERZURUMLUOGLU, Istanbul (TR); Erhan ONAL, Istanbul (TR); Gokhan INCE, Istanbul (TR); Ozgur ATALAY, Istanbul (TR); Asli TUNCAY ATALAY, Istanbul (TR)

(73) Assignees: ISTANBUL TEKNIK UNIVERSITESI, Istanbul (TR); MARMARA UNIVERSITESI, Istanbul (TR)

(21) Appl. No.: 18/842,435

(22) PCT Filed: Mar. 16, 2023
(86) PCT No.: PCT/TR2023/050259

§ 371 Date: Aug. 29, 2024

(30) Foreign Application Priority Data
Mar. 17, 2022 (TR) 2022/004079

(51) Int. Cl. G01L 5/165; A41D 1/00; A61B 5/00; G01L 1/14

(52) U.S. Cl. CPC G01L 5/165; A61B 5/6804; G01L 1/14; A41D 1/002; A61B 2562/0247

(57) ABSTRACT

A textile-based large-area pressure sensing array with a pressure sensor feature includes a pressure plate, a conductive knitted fabric, a dielectric thermoplastic polyurethane layer, and a double-sided fusible layer for use in smart textile, security, healthcare, entertainment, art industries, and robotic applications, and a scalable manufacturing method of the capacitive based pressure sensor arrays for mass production.

### CROSS REFERENCE TO THE RELATED APPLICATIONS

This application is the national phase entry of International Application No. PCT/TR2023/050259, filed on Mar. 16, 2023, which is based upon and claims priority to Turkish Patent Application No. 2022/004079, filed on Mar. 17, 2022, the entire contents of which are incorporated herein by reference.

### TECHNICAL FIELD

The invention is related to a textile-based large-area pressure sensing array having a pressure sensor feature comprising a conductive knitted fabric, a thermoplastic polyurethane layer having dielectric properties and a double-sided fusible layer, and a method of production the array using a pressure plate, for use in smart textile, security, healthcare, entertainment, art industries and robotic applications.

### BACKGROUND

In today's technology, electronic products and textiles have started to be integrated with the production of chips that are small in size, cheap and have low energy requirements. As a result of the studies to provide electrical conductivity to textile structures, electronic textiles have emerged from the electrical reactions of textiles to external stimulus. These electronic textiles have started to turn into systems that can be used by users with multidisciplinary studies in the fields of electronics, computers, control and textiles. It is possible to see electronic textiles in applications in different fields such as sports training, robotics, health imaging, and body motion analysis. There are different types of textile sensors such as resistive, capacitive, optical, inductive, piezoelectric, and solar [1].

Textile-based capacitive sensors are generally designed for pressure, tactile, and strain sensing applications. Textile structures with pressure sensor feature have an important place with their wide range of applications. Textile structures with pressure sensor feature can be used in smart textile, security, healthcare, entertainment, art, and robotic applications. Information on the areas of use are listed below.

Smart textiles: Electronic textiles are suitable for wearable technologies due to their flexible and stretchable properties. The proposed textile-based structures with pressure sensor feature will enable user-computer interactions when used as wristbands or gloves.

Security: The designed textile structures with large-area pressure sensor feature can be used as seat covers that, when integrated with software, will provide observation of posture analysis during driving and prevent accidents by warning the driver when he falls asleep.

Healthcare: Soft pressure sensor arrays have the ability to determine pressure intensity. Therefore, they can be applied to chairs or armchairs to detect sitting/balance disorders. Pressure mats can also be applied to wheelchairs or beds to monitor patient posture, enabling early detection of pressure sores, also known as bedsores.

Entertainment: It can be used to accurately determine the shape of objects placed on it by downsizing the cell size in arrays. Unprecedented games and virtual applications are possible with the help of computer science.

Art: Soft pressure sensors would be a strong candidate for art, instead of using familiar materials such as wood and plastic in musical instruments. For example, the arrays could be designed as piano keyboards and a new instrument could be created with these pressure-sensor keyboards by determining the pressure intensity. In addition, the sensor structure can be used as exercise equipment that can track the steps of dancers and evaluate training progress with a combination of artificial intelligence as good as a professional trainer.

Robotics applications: Pressure sensors can measure different object properties and provide information through the physical interaction between a sensor and an object. For example, a robotic hand picking up an object can be integrated with sensitive sensors. This will provide feedback to the control system that prevents the object from being damaged or released too early. In terms of agriculture, the cost of manual labor for fruit harvesting can be reduced with robotic hands. Robots can recognize and determine if they are mature enough to be picked. Fruit picking robots have already been introduced to the industry. However, fragile fruits can be damaged during picking by grippers. In this situation, soft pressure sensors are really ideal tools to give robotic hands a tactile feeling so that the machines can regulate the pressure applied and not damage the crop.

Some pressure sensors available on the market can measure compressive strength by sensing different pressure points simultaneously. However, some products lack in flexibility and even require a flat surface to be used. To overcome these compatibility issues during use, textile materials are suitable candidates due to their natural, flexible, stretchable, and porous structure. The wearable electronics industry is a highly expanding field and flexibility is an important factor in the wearable electronics industry.

Moreover, production in the former methods is complex, costly, and time-consuming so that not suitable for mass production. The cast silicone approach requires time and constant heat for curing. Some products on the market contain conductive ink or paste released by screen printing or inkjet, which require complex procedures and expensive machinery. In addition, pressure-sensor arrays produced by older methods lack high sensitivity characteristics together with a wide operating range.

Pressure sensors in the known state of the art have been able to show considerable sensitivity in the products on the market using some methods, but a high operating range has not been provided simultaneously. However, thanks to the mentioned invention, the air gaps formed by the pressure plate gaps between the conductive layers and the dielectric layers provide a high degree of sensitivity. Thermoplastic dielectric layers provide the pressure sensor with high repeatability and, most importantly, a wide operating range.

Some pressure sensors on the market contain electrode-like conductive ink or paste released either by screen printing or inkjet printing. In other production techniques, a silicone elastomer is compressed between conductive fabric as a dielectric material. Some examples from the literature are given below:

In the known state of the art with DOI number 10.1016/j.sna.2019.111579, a solution based on polyvinylidene fluoride (PVDF) powder and carbon nanotubes used in the electro-spinning process to create a nanofiber dielectric layer is mentioned. Indium tin oxide polyethylene terephthalate films are deposited as electrodes on the surfaces of the nanofiber dielectric layer.

In the state-of-the-art paper with DOI number 10.1016/j.sna.2020.112029, single-walled carbon nanotube ink and stretchable silver paste were used to create electrode patterns on polyester/spandex (PET/SP) fabrics. Screen printing was used to print single-walled carbon nanotubes on the top and bottom sides of the intermediate fabric. Then the sensors were shaped by laser cutting. Encapsulation pastes were injected into the interlayer. At the end of this step, capacitive pressure sensors with silver/carbon nanotube electrode layers were formed.

In the state-of-the-art paper with DOI number 10.7567/JJAP.57.05GC04, a method of fabricating capacitive air-gap touch sensors by printing and coating is mentioned. The bottom electrode is printed on a PET substrate with silver ink. Then, polydimethylsiloxane was mixed with a curing agent using a polyimide pattern mask. It was spun-coated to form a sheet. The top electrode was formed by spin-coating a stretchable silver ink onto the sheet. The sensor samples were immersed in a tetrabutylammonium (TBAF) bath to remove the layer and form an air gap.

In the state of the art paper with DOI number 10.1002/admt.201700237, a way to increase sensor sensitivity by creating micropores in the dielectric layer is mentioned. For this purpose, silicone elastomers and sugar granules were mixed and cured, then the granules were dissolved in an ultrasonic washing tank, leaving micropores in the silicone elastomers. The electrical layer was compressed between two conductive fabrics.

In the known state of the art paper with DOI number 10.1021/acsami.9b03718, a capacitive sensor enhanced by an oblique microcolumn array structured dielectric layer is mentioned. In this study, the photoresist is covered on a silicon wafer by rotation. It was exposed to UV light to form the template. Poly (dimethylsiloxane) was cast and cured to form a dielectric layer structure with bent micro-column arrays. These were bonded to gold-plated PET electrodes.

In the state of the art paper with DOI number 10.1002/aelm.201870006, a capacitive tactile sensor consisting of graphene electrodes separated by spacers forming air gaps is mentioned.

In the known state of the art paper with DOI number 10.1002/adma.201701218, it is mentioned that a multi-material 3D printing technology was used to produce a haptic sensing mechanism with a special geometry to fit curved surfaces. The bottom electrode layer was printed on a silicon base layer by using silver/silicon ink. Silicone and bio-inks were used to print the insulating and support layers respectively. The upper electrode was printed on the supporting layer to form the sensor.

In known state of the art in United States patent document US2016018274A1, a textile-based pressure sensor with conductive regions and an insulating dielectric spacer between the conductive regions for capacitive measurement of the pressure distribution on a surface is mentioned.

However, despite the advancements in soft capacitive sensor technologies, including the patents and papers cited, there are still significant challenges in terms of manufacturing complexity that limit scalability and operating range in various applications.

The developed textile-based large-area pressure sensing arrays and fabrication technique will enable simple, fast, and relatively inexpensive production.

The developed textile-based large-area pressure sensing arrays are able to detect even low pressures thanks to air-gaps, and thermoplastic polyurethane layers have increased the operating range of the sensor presented here. Therefore, there is a need to develop textile-based large-area pressure sensing arrays and fabrication technique comprising a novel pressure sensor to meet the demand for a scalable, effortless, rapid, and repeatable production strategy for mass production and commercialization.

### SUMMARY

The aim of the present invention is to provide textile-based large-area pressure sensing arrays with a wide operating range and high sensitivity, and a method of production of the sensor arrays.

Another purpose of the present invention is to provide a method of manufacturing textile-based large-area pressure sensing arrays, which makes the manufacturing process of capacitive-based pressure sensors rapid and scalable.

Another purpose of the present invention is to realize the production method of textile-based large-area pressure sensing arrays that enable manufactured sensors to gain sensitivity to pressure as a result of the air gaps formed between the dielectric layers themselves and between the dielectric layers and the conductive fabric layers thanks to the cell-patterned double-sided fusible layers as seen in FIG. 2B.

### BRIEF DESCRIPTION OF THE DRAWINGS

The textile-based large-area pressure sensing arrays realized to achieve the purposes of the present invention and the method of manufacturing these arrays are shown in the attached figure.

These figures are;

FIG. 1: Schematic view of the mat containing the components of textile-based large-area pressure sensing arrays.

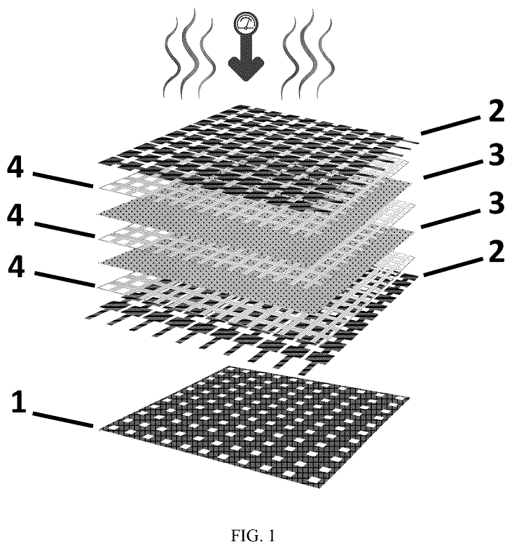

FIG. 2A: A cross-sectional view of the textile-based large-area pressure sensing array developed before the application of heat and pressure, without the application of a pressure plate.

FIG. 2B: A cross-sectional view of a textile-based large-area pressure sensing array developed by applying heat and pressure by the aid of the novel pressure plate

FIG. 2C: A cross-sectional view of a textile-based large-area pressure sensing array without the new pressure plate component developed after heat and pressure are applied.

FIG. 3: A graph comparing the pressure sensitivities of sensors with silicone, fusible layer only and thermoplastic polyurethane (TPU) layer with air gaps, which is the subject of this patent.

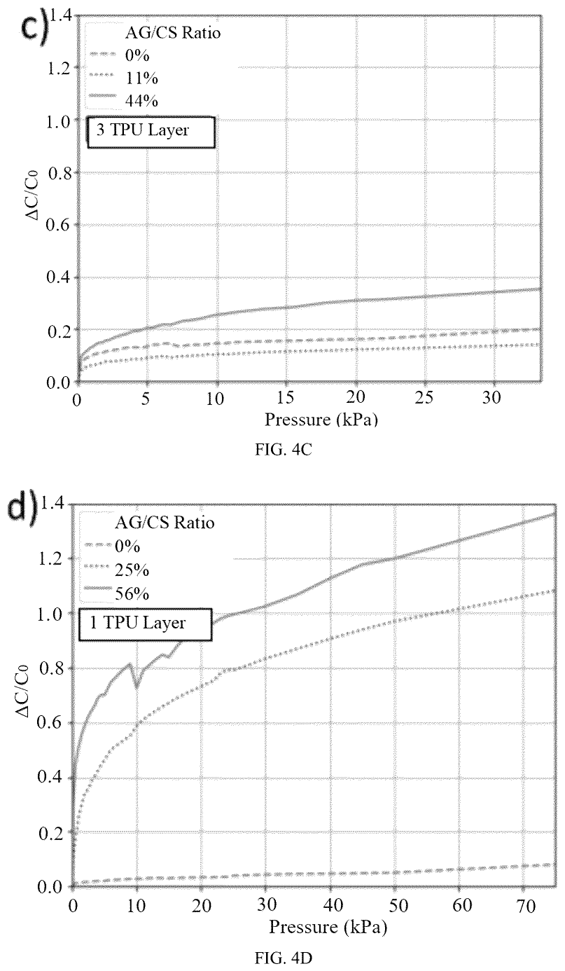

FIGS. 4A-4F: Sensitivity results of square sensor cell samples with one, two and three dielectric thermoplastic polyurethane layers from left to right; FIGS. 4A-4C show sensitivity values of 15×15 mm² pressure sensor cell, FIGS. 4D-4F show graph showing the sensitivity values of a 10×10 mm² sensor cell.

The parts in the figures are numbered individually and the equivalents of these numbers are given below.
5. Pressure plate
6. Conductive knitted fabric
7. Thermoplastic polyurethane film
8. Double-sided fusible layer

### DETAILED DESCRIPTION OF THE EMBODIMENTS

The invention relates to textile-based large-area pressure sensing arrays and includes the following elements:
a pressure plate located under the pressure sensing arrays, with holes responsible for the formation of air-gaps, which are necessary for high sensitivity, and identifying areas that must stick together when heat is applied (1),
conductive knitted fabrics with flexibility and elastic strength (2), which are the layers on the top and bottom sides of the sensor that positioned over the pressure plate (1), where an electrostatic field occurred when a small voltage applied,
thermoplastic polyurethane film (3) with flexible and dielectric (insulating) properties in layers between conductive knitted fabrics (2),
double-sided fusible layers (4) between the layers of thermoplastic polyurethane film (3) and conductive knitted fabrics (2), which ensures the bonding of the layers and the formation of an air gap, and have the ability to melt when heated.

In the known state of the art, production is complex, expensive and time-consuming. These disadvantages are solved by following the proposed layer-by-layer assembly approach. Laser cutting is used to cut the developed textile-based large-area pressure sensing arrays. After the laser cutting, it takes seconds to produce the pressure sensors by pressing with hot plates after mounting the layers on the pressure plate (1). The double-sided fusible layers (4) begin to melt after the heat is applied and the pressure causes the conductive fabric to extend into the pressure plate (1) cavities and air-gaps are formed, resulting in the rapid formation of a textile-based large-area pressure sensing arrays.

Good sensitivity is ensured thanks to the air-gaps created by the pressure plate (1) between the layer of conductive knitted fabric (2) and the layer of thermoplastic polyurethane film (3). The measured sensitivities for the highest performing sample are 306.69×10⁻² kPa⁻¹ (below 1 kPa) and 9.44×10⁻² kPa⁻¹ (between 1 kPa and 30 kPa). As it can be understood from Table 1, when the pressure exceeds 10 kPa, the air gaps inside the sensor are completely closed and the sensitivity of the sensor decreases. At the same time, thermoplastic polyurethane film (3) provides the developed pressure sensor arrays with an increased operating range up to 1000 kPa. These improved textile-based large-area pressure sensing arrays have led to significant improvements in the production of pressure sensors and have enabled the expansion of the use of pressure sensors in many applications.

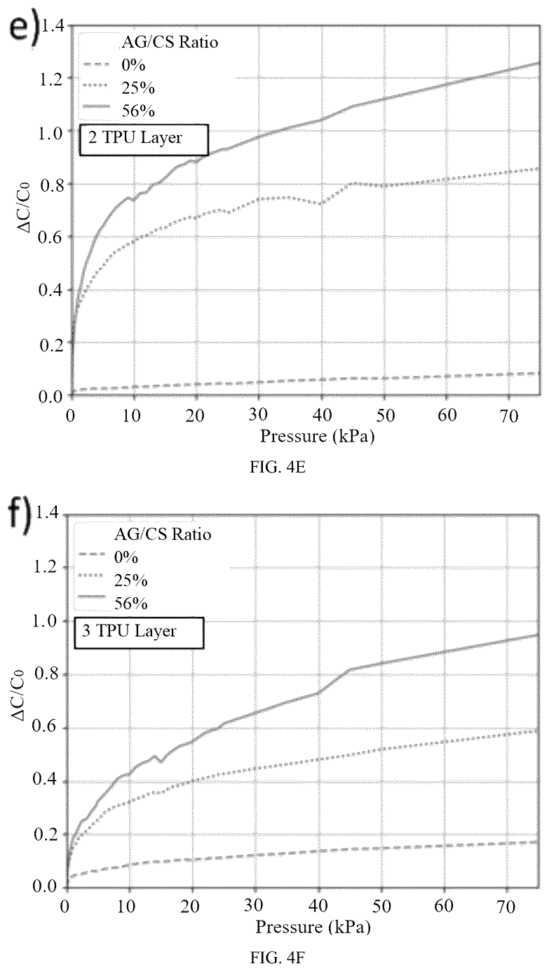

FIGS. 4A-4C show the sensing behavior of a 15×15 mm² sensor cell with an increasing number of thermoplastic polyurethane film (3) layers. FIGS. 4D-4F show the sensing behavior of a 10×10 mm² sensor cell with an increasing number of layers of thermoplastic polyurethane film (3). According to the results of the sensitivity characterization, the pressure sensitivity of the produced sensor cells increases as the amount of air gap increases from 0% to 44%. As it is seen in FIG. 4A, the sensor with the highest air-gap amount and the least amount of thermoplastic polyurethane film layers (3) has the highest sensitivity among the 15×15 mm² sensor cells. In addition, the sensor with no air gap showed the lowest sensitivity performance. As it can be seen in FIG. 4D, the 10×10 mm² sensor cell showed higher sensitivity compared to the 15×15 mm² cell due to the higher air gap/cell size ratio. FIG. 4D-4F indicate that the sensitivity characteristics of the sensor increase with the amount of air gap.

TABLE 1
The pressure sensitivities of the three sensors are compared to analyze the effect of the approaches
on the sensitivity value. (The sensitivity is calculated as S = ΔC/C0/P, where P is the applied
pressure, ΔC and C0 refer to the change in capacitance and initial capacitance value, respectively.)

| | | | | Maximum | | |
|---|---|---|---|---|---|---|
| | Measurement | | Dielectric | pressure | | |
| References | technology | Electrodes | layer | level | | Sensitivity |
| [2] | Capacitance | AgNW/PDMS | MCA | 200 | kPa | 0.28 kPa⁻¹, <10 kPa |
| | | | | | | 0.02 kPa⁻¹, 50-200 kPa |
| [3] | Capacitance | Copper | PDMS | 6 | kPa | 0.1851 kPa⁻¹, 0.5-3 kPa |
| | | binding | | | | 0.7298 kPa⁻¹, up to 6 kPa |
| [4] | Capacitance | ITO-PET | CNT-PVDF | 15 | kPa | 0.99 kPa⁻¹, <1.2 kPa |
| | | | | | | 0.63 kPa⁻¹, 2-15.0 kPa |
| [5] | Capacitance | Conductive | Microporous | 1 | MPa | 121 × 10⁻⁴, <100 kPa |
| | | knitted fabric | silicone | | | 25 × 10⁻⁴, up to 1 MPa |
| (15 × 15 | Capacitance | Conductive | TPU | 444 | kPa | 306.69 × 10⁻², <1 kPa |
| mm²) * | | knitted fabric | | | | 9.44 × 10⁻², 1-30 kPa |
| | | | | | | 0.535 × 10⁻², 30-100 kPa |
| | | | | | | 0.264 × 10⁻², up to 444 kPa |
| (10 × 10 | Capacitance | Conductive | TPU | 1 | mPa | 268.82 × 10⁻², <1 kPa |
| mm²) * | | knitted fabric | | | | 6.36 × 10⁻², 1-30 kPa |
| | | | | | | 0.416 × 10⁻², 30-100 kPa |
| | | | | | | 0.344 × 10⁻², up to 1 MPa |

* These are the 15 × 15 mm² and 10 × 10 mm² versions of the developed textile-based pressure sensor cells.

Air-gaps are created in the cells in the layers to achieve high sensitivity. The flexibility problem of commonly used robust dielectric materials is eliminated by introducing such soft air-gaps between the layers. Even the soft touch of light objects can be detected. Double-sided fusible layers (4) containing cell-like structures were used to bond the layers. These cell-like structures obtained by designing the double-sided fusible layers (4) with a laser cutting machine. Therefore, when heat and pressure are applied, the double-sided fusible layers (4) only adhere to the corners of the cells that helps air-gap formation. A new pressure plate (1) with square cell gaps has been designed for this purpose. The designed pressure plate (1) determines the areas that need to adhere to each other while heat is applied from above to melt the double-sided fusible layer (4), so that the required air gaps can be achieved in exact predetermined locations. The size of the designed pressure plate (1) can be adjusted according to the required pressure sensor shape and cell dimensions.

Although the air gaps in the developed pressure sensing arrays significantly increase the sensitivity of the pressure sensor, the pressure sensing sensitivity loses its effect when the air-gaps are completely compressed under high pressure. In this situation, a layer of thermoplastic polyurethane film (3) helps to extend the sensing range.

The designed pressure plate (1) has holes on it, which are responsible for the formation of air-gaps. During the production process, the pressure plate (1) is placed under the layers. When heat is applied, all double-sided fusible layers (4) start to soften and the pressure plate (1) transmits the pressure only to the edges of the sensor cells. Then the conductive knitted fabric (2) starts to expand through these plate holes. When heat is applied from above to melt the double-sided fusible layers (4), the points of the arrays are determined by the gaps of the designed pressure plate (1), producing air gaps in the cells.

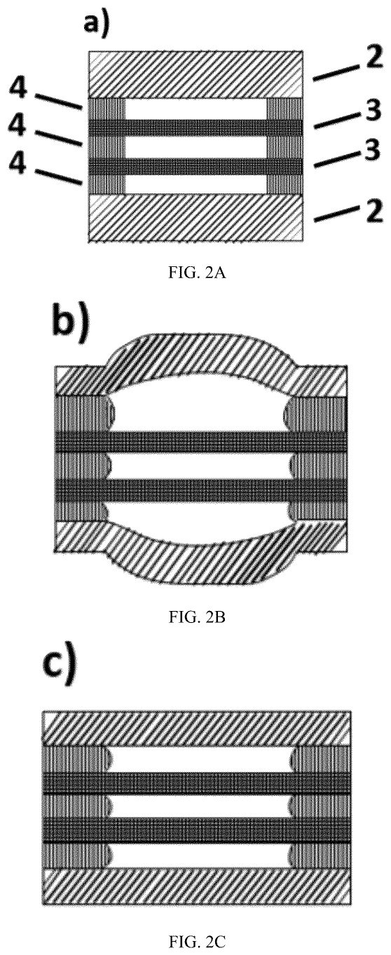

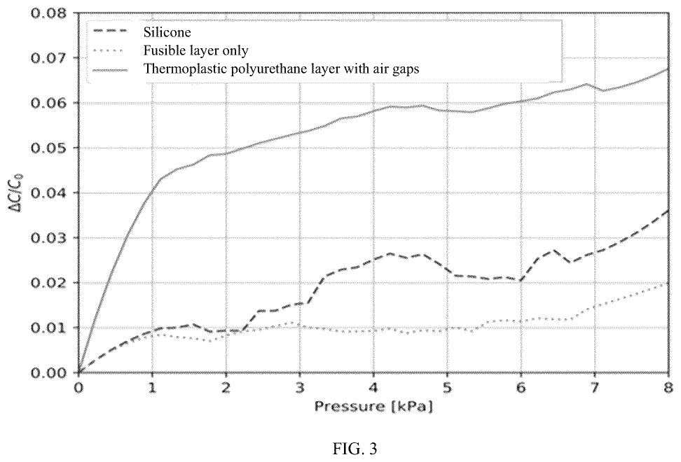

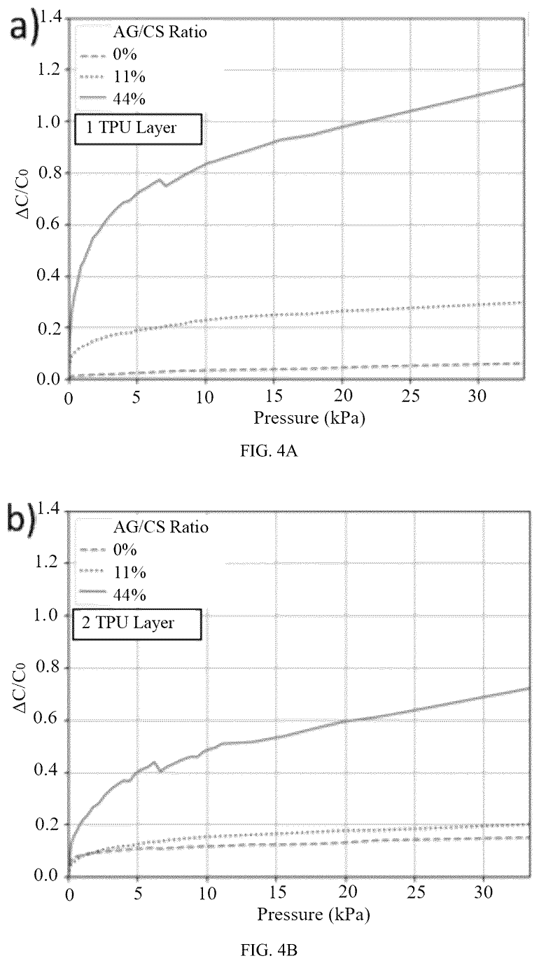

Electrically conductive knitted fabrics (2) were formed into arrays by laser cutting. An electrostatic field is formed when a small voltage is applied to these sensor arrays. Textile-based materials, especially knitted structures, can return to their original shape after being bent, crushed, crumpled or distorted, therefore capacitance can return to initial values and repeatability can be ensured. The conductive knitted fabric (2) has an excellent stretch range and elastic strength. The pressure plate (1) only transmits the applied pressure to the edges of the array cells, while allowing the cell center to extend into the plate cavities. As it can be seen in FIG. 2C, when the process is performed without using the designed pressure plate (1), not only the edges, but the entire layer of conductive knitted fabric (2) loses its natural bulge due to heat and pressure, which adversely affects the formation of air-gaps and sensor performance.

Thermoplastic polyurethane film (3) can stretch up to 500% of its original length, therefore it does not prevent the stretchability of the conductive knitted fabric (2). In this study, thermoplastic polyurethane film (3) was used as the dielectric layer.

The double-sided fusible layers (4) have the ability to melt when heated. The double-sided fusible layers (4) are modified by laser cutting into the cell-like structure so they only stick the edges of the array cells when placed between the layers and let the fabric extend into cavities to form air-gaps. The double-sided fusible layers (4) melt when heat is applied and stick the conductive knitted fabrics (2)/thermoplastic polyurethane film layers (3) together. Air gap formation is also ensured thanks to their special design.

The advantages obtained with the developed sensor arrays are listed below.
Good sensitivity is provided by air-gaps between the layers of conductive knitted fabric (2) and thermoplastic polyurethane film (3).
The thermoplastic polyurethane film (2) provides the sensor with a wide operating range.
It is possible to expand the usage areas of these sensors thanks to the scalable manufacturing technique and the inherent flexibility of textiles.

### REFERENCES

[1] Kadir Özlem, “Bacak hareketi izleme için tekstil tabanl1 alg1lama sistemi”, Yüksek Lisans Tezi, iTÜ, Türkiye, 2018.
[2] Q. Zhou, B. Ji, Y. Wei, B. Hu, Y. Gao, Q. Xu, J. Zhou, B. Zhou, Journal of Materials Chemistry A, 2019, 7, 48 27334.
[3] J. Pignanelli, K. Schlingman, T. B. Carmichael, S. Rondeau-Gagn′e, M. J. Ahamed, Sensors and Actuators A: Physical 2019, 285 427.
[4] X. Yang, Y. Wang, X. Qing, Sensors and Actuators A: Physical 2019, 299 111579.
[5] C. C. Vu, J. Kim, Sensors and Actuators A: Physical 2020, 314 112029.

What is claimed is:

1. A textile-based large-area pressure sensing array, comprising:
conductive knitted fabrics with flexibility and elastic strength, wherein the conductive knitted fabrics are layers in top and bottom sides of the textile-based large-area pressure sensing array placed over a pressure plate, wherein a small voltage is applied to generate an electrostatic field;
the pressure plate placed at a bottom layer of the textile-based large-area pressure sensing array, with holes responsible for formation of air-gaps, which are necessary for high sensitivity, and locate cell edges, wherein the cell edges are configured to stick together when heat is applied,
thermoplastic polyurethane films with flexible and dielectric (insulating) properties in layers between the conductive knitted fabrics, and
double-sided fusible layers, wherein the double-sided fusible layers are located between the layers of conductive knitted fabrics and the thermoplastic polyurethane films, which ensure a bonding of the sensor layers and a formation of air-gaps, and have an ability to melt when heated.
